(19) United States
(12) Patent Application Publication
(10) Pub. No.: US 2019/0240058 A1
(43) Pub. Date: Aug. 8, 2019

(54) Modified male sexual ring

(71) Applicants: David Wright, West Valley City, UT (US); Marquita Lopez, West Valley City, UT (US)

(72) Inventors: David Wright, West Valley City, UT (US); Marquita Lopez, West Valley City, UT (US)

(21) Appl. No.: 15/891,881

(22) Filed: Feb. 8, 2018

(51) Int. Cl. A61F 5/41; A61H 19/00

(52) U.S. Cl. CPC A61F 5/41; A61F 6/04; A61H 19/32

(57) ABSTRACT

The invention is directed to a modified male sexual ring. The ring is intended to fit over the shaft of the penis. The ring is of uniform diameter over the rear half of its length, and tapers gently over the front half of its length to a narrow front end. The ring provides one or more ridges within the interior surface at the rear end to secure a condom, such that the condom may be quickly applied and securely affixed to the ring without slipping off. All edges of the ring are gently rounded to provide maximum comfort.

### CROSS-REFERENCE TO RELATED APPLICATIONS

This application relates back to, and claims the priority of Provisional Patent Application No. 62/457,840 filed on Feb. 11, 2017.

### STATEMENT REGARDING FEDERALLY SPONSORED RESEARCH OR DEVELOPMENT

Not Applicable

### PARTIES TO A JOINT RESEARCH AGREEMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The invention relates generally to sex toys and in particular to a modified male sexual ring. For enhanced female sexual pleasure, the use of a rigid ring or sleeve on the penis to enlarge its diameter is known to be effective. Unfortunately, the existing devices do not provide a means for securing a condom and are less than completely satisfactory in various ways. This is particularly true for devices which are not appropriately rounded or tapered, presenting a hard, sharp edge which can be irritating and even painful for the female partner.

A search of the prior art reveals various devices which have been developed to resolve this problem and similar problems. None are closely related to the present invention, but several include features which resemble those of the present invention. Each has proven to be less than satisfactory for the present purpose in its own way.

Method and apparatus for producing and maintaining a penile erection, U.S. Pat. No. 5,873,813 (priority Feb. 5, 1998), provides an apparatus for producing and maintaining a penile erection in a male suffering from erectile dysfunction. The apparatus includes a resilient ring member defining an inner opening through which the penis is inserted. The inside circumferential surface adjacent the inner opening is supple and adapted to contact the shaft of the penis. The cross-sectional area of the inner opening is adjustable between a minimum cross-sectional area which is sized sufficiently small to restrict the return venous blood flow from the penis to the torso (i.e. restrict excessive venous leakage) and larger cross-sectional areas. The apparatus includes a pair of spaced apart handles extending outwardly from the resilient ring member which allow the user of the apparatus to quickly adjust the cross-sectional area of the inner opening. In use, a flaccid penis is inserted through the inner opening of the resilient ring member and the resilient ring member is placed in the closed position at the base of the penis adjacent the torso. With the resilient ring member in the closed position, the apparatus is moved along the penis shaft away from the torso thereby increasing the amount of engorgement of the portion of the penis on the side of the apparatus opposite the torso. The apparatus is opened and quickly moved again to the base of the penis adjacent the torso. Again, the apparatus is moved along the penis in the closed position away from the torso to incrementally capture more blood within the penis and increase engorgement, thus producing a firm erection suitable for sexual penetration and enjoyment. After a sufficient amount of blood has been captured within the penis to produce a suitable erection, the resilient ring is placed at the base of the penis in the closed position adjacent the torso to maintain the erection.

Sexual enhancement device, U.S. Patent Appl. No. US20020103415A1 (priority Feb. 1, 2001), provides a versatile vibrating sexual enhancement device for use by either men or women individually, or by both during sexual intercourse. The device comprises a phallic portion that contains a vibrator therein and a loop portion comprised of an elastomeric material. The loop portion is configured to encircle erect penises of various sizes without causing circumferential constriction thereto, thereby being comfortable as well as being easy to install and remove. Also, the loop portion is slidable on various sized penises, which even further increases the utility of the device during different sex acts. The device includes a controller that serves to turn the vibrator on and off. In certain embodiments the controller is remote from the device being connected thereto by a pair of electrical wires. In other embodiments the controller is located within the phallic portion of the device.

Condom restrainer ring structure with retention notch, U.S. Pat. No. 5,370,131 (priority Aug. 3, 1993), provides a condom retainer ring structure for use on a condom, which has an elongated thin-walled tubular sheath of resilient material closed at one end and open at the other end, with a bead surrounding the opening. The condom retainer ring structure has a first ring for positioning at the periphery of the opening, adjacent the bead that surrounds the sheath. A second ring is connected to and adjacent the first ring. A bridge having a retention notch therein joins the rings generally in a single plane when the retainer ring structure is not in use and in an unflexed condition. In use, when a condom is worn on the penis of a user, the first ring surrounds the sheath on the shaft of the penis, the second ring encircles the scrotum to anchor the condom, and the bridge is flexed thereby, constricting the retention notch. The retention notch, preferably positioned over the bead of the condom, grabs the bead to retain the condom.

Erection support ring, U.S. Patent Appl. No. US20030083598A1 (priority Oct. 31, 2001), provides an erection support ring which includes a ring body, fabricated of soft rubber such as copolymerized rubber, swollen elastomer, or silicone rubber, and having an insert hole that elastically constricts a penis, and a cavity into which a small vibrator is removably placed. The ring body has a projection integrally formed therewith projecting in a direction in parallel with the center axis of the insert hole of the ring body. The small vibrator housed in the cavity vibrates, thereby providing stimulation at a comfortable level to the penis, leading the penis to erection, maintaining the erection of the penis, and finally inducing the penis to ejaculation. The vibration of the small vibrator is transferred to female sexual organs, thereby contributing to enhancing sexually pleasurable sensation to both of the male and the female.

Some of the prior art inventions present certain disadvantages. The present invention has been developed for the purpose of addressing and resolving these disadvantages, by providing an improved device. A modified male sexual ring, which tapers gently at the front end and provides one or more ridges at the rear end to secure a condom, such that the condom may be quickly applied and securely affixed to the ring without slipping off, would resolve these problems.

### SUMMARY OF THE INVENTION

Accordingly, the invention is directed to a modified male sexual ring. The ring is intended to fit over the shaft of the penis. The ring is of uniform diameter over the rear half of its length, and tapers gently over the front half of its length to a narrow front end. The ring provides one or more ridges within the interior surface at the rear end to secure a condom, such that the condom may be quickly applied and securely affixed to the ring without slipping off. All edges of the ring are gently rounded to provide maximum comfort.

Additional features and advantages of the invention will be set forth in the description which follows, and will be apparent from the description, or may be learned by practice of the invention. The foregoing general description and the following detailed description are exemplary and explanatory and are intended to provide further explanation of the invention.

### BRIEF DESCRIPTION OF THE DRAWINGS

The accompanying drawings are included to provide a further understanding of the invention and are incorporated into and constitute a part of the specification. They illustrate one embodiment of the invention and, together with the description, serve to explain the principles of the invention.

FIG. 1 shows a rear perspective view of the first exemplary embodiment, displaying the ring 10, the interior surface 11, and the ridges 12.

FIG. 2 shows a side view of the first exemplary embodiment, displaying the ring 10, the rear half 13, and the front half 14.

FIG. 3 shows a rear view of the first exemplary embodiment, displaying the ring 10, and the ridges 12.

FIG. 4 shows a front view of the first exemplary embodiment, displaying the ring 10, and the front half 14.

FIG. 5 shows a side perspective view of the first exemplary embodiment, displaying the ring 10, the interior surface 11, the rear half 13, and the front half 14.

FIG. 6 shows a rear perspective view of the first exemplary embodiment, displaying the ring 10, the interior surface 11, the ridges 12, and the rear half 13.

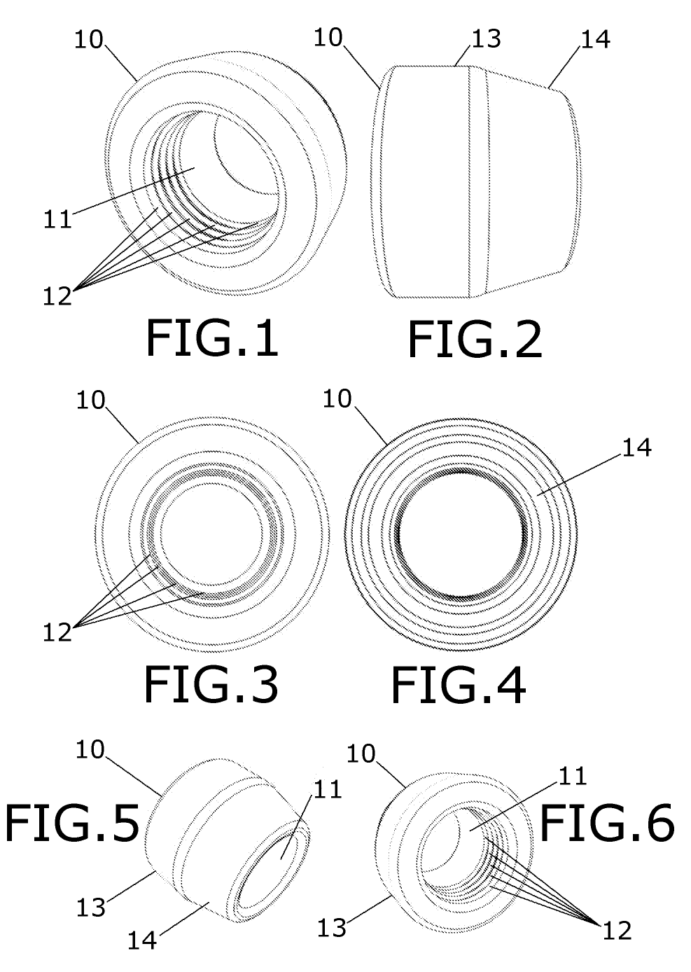

### DETAILED DESCRIPTION OF THE INVENTION

Referring now to the invention in more detail, the invention is directed to a modified male sexual ring 10.

The first exemplary embodiment provides a ring 10 which is intended to fit over the shaft of the user's penis. The ring 10 is of uniform diameter over the rear half 13 of its length, and tapers gently over the front half 14 of its length to a narrow front end. The ring 10 provides one or more ridges 12 within the interior surface 11 at the rear end to secure a condom, such that the condom may be quickly applied and securely affixed to the ring 10 without slipping off.

The ring 10 may be worn with or without a condom, may be used in conjunction with other sex toys, and is best applied before the user becomes sexually aroused. All edges of the ring 10 are gently rounded to provide maximum comfort.

The ring 10 is preferably 2 inches in length, with the interior surface 11 providing an interior diameter of 1.76 inches and the ridges 12 providing an interior diameter of 1.6 inches. Larger and smaller sizes may be provided.

To use the first exemplary embodiment, the user slides the ring 10 over the penis such the ridges 12 are close to the base of the penis. Optionally, the user may then cover the penis and the ring 10 with a condom, tusking the rear edge of the condom under the ridges 12 to secure it in position.

The ring 10 is preferably manufactured from a rigid, durable material which is corrosion resistant and easily cleaned, such as stainless steel, plastic, brass, or aluminum alloy. Components, component sizes, and materials listed above are preferable, but artisans will recognize that alternate components and materials could be selected without altering the scope of the invention.

While the foregoing written description of the invention enables one of ordinary skill to make and use what is presently considered to be the best mode thereof, those of ordinary skill in the art will understand and appreciate the existence of variations, combinations, and equivalents of the specific embodiment, method, and examples herein. The invention should, therefore, not be limited by the above described embodiment, method, and examples, but by all embodiments and methods within the scope and spirit of the invention.

I claim:

1. A modified male sexual ring, intended to fit over the shaft of the penis, which is of uniform diameter over the rear half of its length, and tapers gently over the front half of its length to a narrow front end.

2. The ring of claim 1, wherein the ring provides one or more ridges within the interior surface at the rear end to secure a condom, such that the condom may be quickly applied and securely affixed to the ring without slipping off.

3. The ring of claim 1, wherein all edges of the ring are gently rounded to provide maximum comfort. The ring of claim 1, wherein

4. The ring of claim 1, wherein the ring may be worn with or without a condom, may be used in conjunction with other sex toys, and is best applied before the user becomes sexually aroused.

5. The ring of claim 1, wherein the ring is preferably 2 inches in length, with the interior surface providing an interior diameter of 1.76 inches and the ridges providing an interior diameter of 1.6 inches.

6. The ring of claim 1, wherein the user slides the ring over the penis such the ridges are close to the base of the penis. Optionally, the user may then cover the penis and the ring with a condom, tusking the rear edge of the condom under the ridges to secure it in position.

7. The ring of claim 1, wherein the ring 10 preferably manufactured from a rigid, durable material which is corrosion resistant and easily cleaned, such as stainless steel, plastic, brass, or aluminum alloy.
